(19) United States
(12) Patent Application Publication
(10) Pub. No.: US 2024/0415683 A1
(43) Pub. Date: Dec. 19, 2024

(54) APPARATUS AND METHOD FOR CREATING A PLICATION IN AN INTERNAL ORGAN

(71) Applicant: BariaTek Medical, Paris (FR)

(72) Inventors: Youssef BIADILLAH, Maisons-Laffitte (FR); Yonatan GRAY, Paris (FR); Christophe NAZ, Fontainebleau (FR)

(73) Assignee: BariaTek Medical, Paris (FR)

(21) Appl. No.: 18/706,929

(22) PCT Filed: Nov. 3, 2022
(86) PCT No.: PCT/EP2022/080654

§ 371 Date: May 2, 2024

(30) Foreign Application Priority Data
Nov. 3, 2021 (EP) 21315222.6

(51) Int. Cl. A61F 5/00; A61B 17/00; A61B 17/12

(52) U.S. Cl. CPC A61F 5/0089; A61B 17/12013; A61F 5/0069; A61F 5/0076; A61B 2017/00818

(57) ABSTRACT

Apparatus (10) for forming a plication (64) in a wall of internal tissue of a patient. The apparatus (10) comprises an elongated instrument (11) for insertion into a patient close to the tissue wall, optionally through a natural body opening. The apparatus (10) comprises one or more of (a) a traction device (20) configured to engage tissue of the wall and to pull on tissue of the wall to create a protrusion of wall tissue; (b) a ligating device (30) configured to at least partially circumscribe the or a protrusion in tissue of the wall to form the plication (64) in the tissue wall; (c) a fixation device (40) for fixing the plication (64) in the tissue wall.

This application is a National Stage completion of PCT/EP2022/080654 filed Nov. 3, 2022, which claims priority from European patent application serial number 21315222.6 filed Nov. 3, 2021.

### FIELD OF THE INVENTION

The present invention relates to the field of medical interventions that include forming a plication in a wall of internal tissue. Some non-limiting aspects relate to interventions on an internal organ such as in the digestive system, for example, in the gastro-intestinal tract, and optionally the stomach. Other non-limiting aspects relate to interventions on tissues comprising a parietal layer of muscle tissue.

Formation of a plication in a stomach wall may be one technique for treating certain conditions relating to the digestive system. However, technical challenges remain in performing such a procedure, especially from within the stomach itself.

### SUMMARY OF THE INVENTION

Broadly speaking, a first aspect of the invention provides apparatus for forming a plication in a wall of internal tissue of a patient. The apparatus includes an elongated instrument for insertion into a patient, optionally through a natural body opening, close to the tissue wall. The apparatus comprises one or more of:
(a) a traction device configured to engage tissue of the wall and to pull on tissue of the wall to create a protrusion of wall tissue; and/or
(b) a ligating device configured to at least partially circumscribe the or a protrusion in tissue of the wall to define and/or form the plication in the tissue wall; and/or
(c) a fixation device for fixing the plication in the tissue wall.

Specific aspects of the invention provide a combination of (a) and (b); and/or a combination of (b) and (c); and/or a combination of (a) and (c); and/or a combination of (a), (b) and (c).

The wall may be a wall of an internal organ of the patient, and references to the wall may be an organ wall. Additionally or alternatively, the wall may be a tissue wall of the digestive system, for example, of the gastro-intestinal tract. Additionally or alternatively, the wall may be a parietal wall. Additionally or alternatively, the wall may be a wall including muscle tissue. One example of all of the above is a wall of the stomach, and references to the wall may be a stomach wall.

The traction device, if used, may comprise a suction or aspiration device for pulling on wall tissue by application of negative pressure. Alternatively, the traction device may comprise a mechanical engagement device, for example, gripper jaws for gripping wall tissue, or a helical penetration device that is removably screwable into the wall tissue.

The traction device may be extendable and/or retractable (for example with respect to a main body of the instrument and/or with respect to the ligating device) to engage wall tissue and to perform the pulling operation to create the wall tissue protrusion.

Additionally or alternatively to any of the above, the ligating device, if used, may comprise a ligating element, for example, a ligature. The ligating element may have contractible loop. The loop may be a closed loop, or the ligating element may comprise a loop region of an elongated element. The ligating device and/or element may comprise a lasso or a snare. The ligating device optionally does not penetrate through the tissue.

The ligating device may serve to at least partly immobilize the protrusion of wall tissue and/or to laterally contract the protrusion of wall tissue.

In some embodiments, the ligating element circumscribes the traction device in a circumferential direction, at least in one operative condition of the ligating device and/or of the traction device. Additionally or alternatively, the traction device may extend through a loop of the ligating device, at least in one operative condition of the ligating device and/or of the traction device.

Additionally or alternatively to any of the above, the fixation device, if used, may comprise a tissue-penetrating fixation element, and/or an insertion device for inserting the or a fixation element, the fixation element being preferably configured to extend through the wall tissue at the plication. The insertion device may include a tubular needle having a sharp end for penetrating the wall tissue to create a passage for the fixation element. The fixation element may be housed at least partly within the hollow of the tubular needle, for example, in a non-deployed configuration. The fixation element may be deployable (e.g. self-deployable and/or self-expandable) by ejecting the fixation element from the needle, and/or by retracting the needle to expose the fixation element. The insertion device may optionally be loaded with multiple fixation elements for deployment sequentially, to enable multiple fixation elements to be deployed, for example, at multiple positions, without having to remove the apparatus from the body each time.

In some embodiments, the insertion device and the ligating device are carried on the same carrier. The ligation device and the insertion device may be arranged on the carrier adjacent one another for interacting with the same region, or neighbouring regions, of tissue of the wall. The carrier may be a movable part of the instrument. Additionally, the carrier may be a tube distinct from the traction device. The tube may be extendable and/or retractable. Additionally or alternatively, the tube may be shaped with an angle to present the ligating device and/or the insertion device at an angle to the traction device, for example, generally orthogonally to the traction device and/or generally orthogonal to the pulling direction of the traction device.

Additionally or alternatively to any of the above, the instrument may further comprise an elongated main body having a working end for contacting the tissue wall, and an operator end from which one or more of the traction device, the ligating device and/or the insertion device may be operated (and/or manipulated and/or actuated). In some embodiments, the main body includes a first channel for accommodating the traction device and/or through which the traction device may be operated. The main body further includes a second channel for accommodating a carrier tube for the ligating device and/or the insertion device.

The instrument may be insertable into the interior of, for example, the body organ, and the protrusion of organ wall tissue may be an inward protrusion. In some embodiments, the organ is an organ of the digestive system, for example, an organ of the gastro-intestinal tract. The organ may be the stomach, and the instrument may be insertable through the patient's mouth to access the stomach wall.

The tissue-penetrating fixation element may comprise a first shoulder for anchoring against a first side of tissue (e.g. dual tissue walls of the plication), a second shoulder for anchoring against a second side of tissue, and a connecting element extending between the first shoulder and the second shoulder. In use, the connecting element extends through one or more through-apertures from the first side of the tissue plication to the second side. The first anchor resists migration of the anchor from the first side to the second, and the second should resists migration of the anchor from the second side to the first.

The connecting element may, for example, be elastic and/or pseudoelastic. The connecting element may be biased to a first length, but be longitudinally extendable. The connecting element may maintain a bias tending to draw the dual tissue walls into face to face relation to fix the plication. Elastic and/or pseudoelastic extension may also accommodate a range of tissue wall thicknesses at the plication, and also reduce risk of injury to the tissues or breakage of the fixing element in the case of occasional excessive forces and/or muscular contractions occurring at the plication.

The fixation element may be inserted in a non-deployed configuration in which the first and second shoulders are configured in a position with a smaller outer diameter, in particular by being rotated or folded to be generally flat and/or in line with the connecting element. The shoulders may be deployed, e.g. sequentially, to their projecting positons.

In a closely related second aspect, the invention provides a method of operation of an apparatus for forming a plication in a wall of internal tissue of a patient, the apparatus comprising an elongated instrument for insertion into a patient, the apparatus optionally according to the first aspect, and optionally including any of the features described above. The method comprises one or more of:
(a) operating a traction device to engage with tissue of the wall and to pull on tissue of the wall to create a protrusion of wall tissue; and/or
(b) operating a ligating device to circumscribe the or a protrusion in wall tissue to define and/or form the plication in the wall; and/or
(c) operating a fixation device to fix the plication in position.

Specific aspects of the method provide a combination of (a) and (b); and/or a combination of (b) and (c); and/or a combination of (a) and (c); and/or a combination of (a), (b) and (c).

In a closely related third aspect, the invention provides a method for creating a plication in a wall of internal tissue of a patient, optionally using an apparatus according to the first aspect and optionally using any of the features of the first aspect described above, and/or optionally using a method of operation according to the second aspect and optionally using any of the features of the second aspect described above. The method comprises or further comprises:
inserting an instrument into the patient through a natural or non-natural opening, to the tissue wall; and one or more of the following:
(ii) engaging tissue of the wall, and pulling on the tissue to create a protrusion of wall tissue; and/or
(iii) ligating the or a protrusion of wall tissue, to circumscribe the protrusion and define and/or form the plication in the tissue wall; and/or
(iv) fixing the plication using a tissue-penetrating fixation element.

Specific aspects of the method provide a combination of (ii) and (iii); and/or a combination of (iii) and (iv); and/or a combination of (ii) and (iv); and/or a combination of (ii), (iii) and (iv).

As with the first aspect, in the method of the second aspect and/or third aspect, the wall may be a wall of an internal organ of the patient, and references to the wall may be an organ wall. Additionally or alternatively, the wall may be a tissue wall of the digestive system, for example, of the gastro-intestinal tract. Additionally or alternatively, the wall may be a parietal wall. Additionally or alternatively, the wall may be a wall including muscle tissue. One example of all of the above is a wall of the stomach, and references to the wall may be a stomach wall.

The methods of the second and third aspects may optionally further include the step of releasing a pulling device from the tissue wall at least once the tissue protrusion has been ligated (and optionally before or after fixation of the plication, for example, by a tissue-penetrating fixation element). Additionally or alternatively, the methods of the second and third aspects may optionally further include the step of releasing a ligating device at least once the plication has been fixed, for example, using a tissue-penetrating fixation element.

Closely related fourth, fifth and sixth aspects of the invention relate to fixing a functional device with respect to a stomach of a patient, optionally for the treatment of diabetes or obesity. The functional device may be configured for occupying and/or reducing stomach volume, and/or for affecting digestion of food. The functional device may, for example, be or comprise a bypass sleeve.

The fourth aspect provides a method of placing a functional device in the stomach of a patient, the method comprising:
forming a plication in wall tissue of the stomach, the plication serving as a mounting site for the functional device; and
placing the functional device such that the functional device is mounted with respect to the stomach using, at least partly, the plication.

The plication may define one or more of a seat, a dock, a reinforced section of tissue wall, to serve as the or a mounting site for the functional device. The plication may extend in a line, optionally a continuous line, for example, an annular form providing a closed-loop shape for the mounting site. By using the stomach wall tissue itself to form the mounting site, the functional device can be reliably mounted in the stomach, and also placed and/or removed easily, without having to insert a separate large dock member to define a mounting site in the stomach wall.

The step of forming the plication may optionally include the method of the second and/or third aspect above.

The fifth aspect similarly provides apparatus for placing a functional device, such as a bypass sleeve, within a stomach of a patient, the apparatus comprising:
first apparatus for forming a plication in wall tissue of the stomach, the plication serving as a mounting site for the functional device; and
second apparatus for placing the functional device such that the functional device is mounted with respect to the stomach using, at least partly, the plication.

The fifth aspect may optionally use the apparatus of the first aspect above, and/or the method of one or more of the second, third and fourth aspect above. The first and second apparatus may optionally form part of the same unit (optionally using one or more parts in common), or they may be separate apparatus.

The sixth aspect provides a functional device for placement at least partly within a stomach of a patient. The functional device may optionally be configured for occupying and/or reducing stomach volume, and/or for affecting digestion of food. In some embodiments, the functional device is or comprises a bypass sleeve.

The functional device comprises a mounting portion configured to be mounted to a plication formed in wall tissue of the stomach, whereby the functional device is mountable with respect to the stomach using, at least partly, the plication.

The mounting portion may, for example, comprise an annular portion for example, an annular mouth, for engaging an annular plication in stomach wall tissue.

Although certain ideas, features and aspects have been presented above, the invention may also be defined more broadly and in other aspects. Protection is claimed for any novel feature and/or idea described herein and/or illustrated in the drawings, whether or not emphasis has been placed thereon.

### BRIEF DESCRIPTION OF THE DRAWINGS

FIG. 1 is a schematic longitudinal half-section view and front view of an apparatus for creating a plication in an internal organ of a patient.

FIG. 2 is a schematic side view of an apparatus for creating a plication in an internal organ of a patient.

FIG. 3 is a schematic inside view of an apparatus for creating a plication in an internal organ of a patient.

FIG. 4 is a schematic side view and detailed view of elements from an embodiment of an apparatus for creating a plication in an internal organ of a patient.

FIG. 5 is a schematic perspective view of an embodiment of apparatus for creating a plication in an internal organ of a patient.

FIGS. 6a-6h are schematic diagrams illustrating steps in an example technique for forming a plication in a stomach wall.

FIG. 7 is a schematic section illustrating a first example of elongated plications formed in a stomach wall.

FIG. 8 is a schematic section illustrating a second example of elongated plication formed in a stomach wall to define a mounting site for a functional device.

### DETAILED DESCRIPTION OF PREFERRED EMBODIMENTS

Non-limiting embodiments of the invention are now described by way of example only. In the illustrations, the same reference numerals are used to denote the same or equivalent features amongst different embodiments and examples. Unless described to the contrary, the description of a feature in one embodiment may also apply to the same or equivalent feature in one embodiment or example. Features may also be interchanged in embodiments as desired.

A plication is a fold in, for example, an originally smooth tissue wherein a first layer and second layer of tissue are arranged to at least partially overlay one another whilst remaining attached to one another at the fold. The layers may be at least partially in contact with one another or they may be at least partially separate. The overlaying of a first layer and a second layer of tissue forms the dual tissue walls of a plication.

The term tissue is used to refer to an internal bodily structure of cells forming a body structure in which a plication is possible, other terms to be considered equivalents may be, for example, organ wall; tissue wall; parietal tissue; organ tissue, internal tissue. All of these terms may be used interchangeably to understand the following invention.

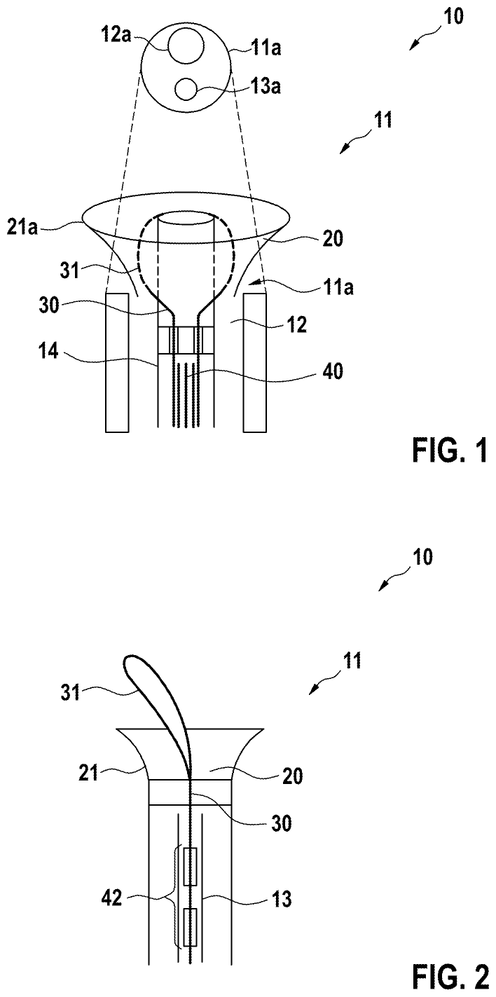

Referring to FIGS. 1 to 4 an apparatus 10 configured for creating a plication in a wall of an internal organ of a patient. The apparatus 10 may comprise an instrument 11 and may further comprise or use one or any combination of: a traction device 20; a ligating device 30; a fixation device 40.

A first aspect of the invention provides an apparatus presenting: a combination of a traction device 20 and a ligating device 30; a combination of a traction device 20 and a fixation device 40; a combination of a ligating device 30 and a fixation device 40; a combination of a traction device 20, a ligating device 30 and a fixation device 40.

In some embodiments, an apparatus 10 for creating a plication is configured for introduction into a patient's body; optionally it may be suitable for introduction through a natural body opening.

In some embodiments the instrument 11 may provide an elongated main body having a working end 11a for contacting the tissue wall and an operator end 11b configured to operate, optionally manipulate, at least one or more of: a traction device 20; a ligating device 30; a fixation device 40. The working end may be provided at a proximal end 11a of the instrument 11 and the operator end may be provided at a distal end 11b of the instrument 11. The operator end 11b may be configured for an operator to operate; manipulate; actuate the working end 11a.

In other words, a means for manipulating the apparatus 10, optionally for operating the devices 20; 30; 40 may be provided, for interaction with a tissue wall at a proximal end 11a when the distal end 11b is operated. The instrument 11 of the apparatus 10 may provide at least one internal channel 12 extending at least part of the length of the instrument 11 and may be configured to accommodate a traction device 20, optionally the traction device 20 may be operated through the channel 12. The instrument 11 may further provide a second channel 13 that may be configured so as to accommodate a carrier tube 14 that may comprise a ligating device 30, additionally or alternatively, the carrier tube may accommodate a fixation device 40. The channel 12 may be configured to further accommodate a ligating device 30, additionally or alternatively a fixation device 40. The instrument 11 may provide a plurality of channels for individually accommodating the devices 20; 30; 40.

In some embodiments the instrument 11 may be configured for insertion into the interior of a body organ and the plication created may be an inward protrusion of an internal tissue wall. The instrument may be at least partly flexible for adapting to a natural passageway for entering a patient, for example through the digestive tract. In some embodiments the tissue may be a tissue of the digestive system, for example, a tissue, optionally an organ, of the gastro-intestinal tract. The organ may be, for example, the stomach and the instrument 11 may be, for example, inserted through a patient's mouth to access the stomach wall.

In some embodiments, the traction device 20, if provided, may extend along a channel 12, optionally extending from a distal end 11b toward a proximal end 11a of the instrument 11 and may comprise a means for engaging with a tissue wall situated at a proximal end 11a, for example, a suction or aspiration device 21 configured to pull on the tissue wall by exerting negative pressure on the organ wall. Optionally, the suction or aspiration device, if provided, may comprise a tapered profile 21a at the distal end of the instrument 11 and configured for guiding the fold in an organ wall, further optionally wherein the tapered profile of the suction or aspiration device is removable from the instrument 11.

Additionally or alternatively the traction device 20 may provide a mechanical device at a distal end for attaching to the tissue of an organ wall. The mechanical device may be, for example, gripping jaws that are configured to claps onto the organ wall or a helical penetrating device configured to removably screw into an organ wall.

The traction device 20 may be configured to be extendable with respect to the main body of the instrument 11 to engage with an organ wall. Optionally it may be extendable with respect of the ligating device 30. Additionally or alternatively, the traction device 20 may be configured to be retractable with respect to a main body of the instrument 11. Optionally it may be configured to be retractable with respect of a ligating device 30. The traction device 20 may be further configured to perform a pulling operation, optionally to create a protrusion of the tissue it engages with.

In other words the traction device 20 may extend at least a part of the length of an instrument 11 and may be accommodated by a channel 12, optionally a channel 13, of the instrument 11. The traction device 20 may be operable by a means of control situated at a distal end 11b of the instrument 11. Operation of the means of control may induce one or more of or all of: attaching or engaging a traction device to a tissue wall; longitudinally pulling a tissue; creating or guiding a protrusion in the wall; creating or guiding a fold in wall.

The traction device 20 facilitates attaching the apparatus 10 to the tissue wall and creating a protruding fold in the tissue. The aspirating element, if provided, permits a controlled portion selection of the tissue, thus allowing for at least partly homogenous or same sized plications in the case that multiple plications are performed.

In some embodiments the apparatus may comprise a carrier 14 configured to extend at least a part of the length of a channel, for example, 12; 13, optionally extending from a distal end 12b; 13b, toward a proximal end 12a; 13a of an instrument 11.

The carrier 14 may be configured to carry at least one, optionally both, of a ligating device 30 and a fixation device 40. Additionally or alternatively, if both provided, a ligation device 30 and a fixation device 40 may be generally adjacent one another for interaction with a same region, optionally for interacting with neighbouring regions, of an organ wall tissue.

In some embodiments, the carrier 14 may be configured to be moveable, additionally; it may be a tube, optionally a tube distinct from the traction device 20. The carrier may further be longitudinally extendable with regards of the instrument 11.

Additionally or alternatively, the carrier 14 may be shaped with an angle so as to present, either or both, the ligating device 30 and the fixation device 40 at an angle to the traction device 20, for example, generally orthogonally to the traction device 20, additionally or alternatively, generally orthogonally to the pulling direction of the traction device 20.

Further additionally or optionally, in some embodiments the carrier 14, optionally the tube of the carrier, may be at least partly flexible and/or configured to at least partially bend so as to perform an angle so as to present, either or both, the ligating device 30 and the fixation device 40 at an angle to the traction device 20, for example, generally orthogonally to the traction device, additionally or alternatively, generally orthogonally to the pulling direction of the traction device 20.

The ligating device 30 may be configured for circumscribing a protrusion of wall tissue. The ligating device 30 may extend at least a part of the length of an instrument 11, optionally extending from a distal end 11b toward a proximal end 11a of an instrument 11, further optionally extending from a distal end 13b toward a proximal end 13a of a channel 13. Channel 13 extending at least a part of the length of the instrument 11. The ligating device 30 may provide a shaft 30′ that may extend the length of a channel 12 or 13. The ligating device 30 may be further configured to be extendable, additionally or alternatively retractable, with respect to a main body of an instrument 11, additionally or alternatively with respect to a traction device 20.

The ligating device 30, if provided, may comprise a ligating element 31, for example, a ligation, configured to at least partially circumscribe a protrusion of an internal tissue. The ligating element 31 may provide a loop, optionally a contractible or adjustable loop, further optionally a closed loop. Additionally or alternatively the ligating element 31 may comprise a loop region of an elongated element. The ligating element 31, optionally the ligating device 30, may be, for example, a lasso or a snare.

In some embodiments the ligating element 31 may be at least partly rigid, additionally or alternatively at least partly pliable; optionally it may be made of any suitable material such as a metal, optionally a metal alloy. It may, for example, be made of Nitinol.

The ligating device 30, if provided, may be further configured to at least partly immobilize the protrusion, and/or to laterally contract a protrusion of an organ wall.

In other words the ligating device 30 may facilitate the immobilizing a protrusion in a tissue wall with regards of the surrounding tissue. The ligating device 30 may be further configured to contract a first layer of a fold of tissue toward a second layer of a fold of tissue, optionally wherein the two layers are contracted so as to be in contact with one another.

In some embodiments the ligating element 31 may be configured to circumscribe the traction device 20, optionally in an operative condition of the ligating device 30. Additionally or alternatively the ligating element 31 may circumscribe the traction device 20 at least in one operative condition of the traction device.

In some embodiments, the traction device 20, if provided, may be configured to extend through a loop of the ligating device 30, at least in one operative condition of the ligating device 30, additionally or alternatively in an operative condition of the traction device 20.

In other words the ligating device 30 may extend at least a part of the length off an instrument 11 and may be accommodated by a channel 13, optionally a channel 12, of the instrument 11. The ligating device 30 may be operable by a means of control situated at a distal end of the instrument 11. Operation of the means of control may induce one or more of or all of: a circumscription of a protrusion in a parietal tissue; an immobilization of a protrusion in an internal tissue; a lateral contraction of a protrusion in a tissue wall.

The above mentioned characteristics of the ligation device 30 allow for an improved apparatus for generating a plication, for example, in an internal organ of a patient. The circumscription and immobilization of the site where a plication is desired may help to avoid the displacement of the tissue during the placing of fixation elements. Bringing the two layers forming the plication into contact may allow for better anchoring of fixation means, thus there is a reduced chance of the fixing elements dislodging.

The fixation device 40 may be provided for fixing a plication in an organ wall. The fixation device 40 may be configured to extend at least a part of the length of a channel 12 or 13. Optionally extending from a distal end 12b; 13b toward a proximal end 12a; 13a of the respective channels 12 and 13. Channels 12 and 13 may optionally extend at least a part of the length of an instrument 11, further optionally extending from an operating distal end 11b toward a working proximal end 11a of an instrument 11. The fixation device 40 may be further configured to be extendable, additionally or alternatively retractable, with respect to a main body of an instrument 11, additionally or alternatively with respect to a traction device 20.

The fixation device 40 may provide an insertion device 41 that may be configured to introduce at least one tissue penetrating element through the plication. The insertion device 41 may be arranged so as to create a passageway or through-aperture in the tissue wall for insertion of a fixation element 42. The insertion device 41 may comprise a tubular needle that may have a sharp end configured to penetrating at least one layer of the plication, optionally configured to deliver a fixation element 42. The tubular needle may provide a hollow passage for at least partially housing at least one, optionally a plurality of fixation elements 42, further optionally the fixation element 42 in the hollow may be in a non-deployed state.

In other words the fixation device 40 may extend at least a part of the length of an instrument 11 and may be accommodated by a channel 13, optionally a channel 12, of the instrument 11. The fixation device 40 may be operable by a means of control situated at a distal operating end 11b of the instrument 11. Operation of the means of control may induce one or more of or all of: penetration of an internal tissue creating a through-aperture; insertion of a fixation element 42 by means of an insertion device 41; ejection of a fixation element from within an insertion device; deployment of a fixation element by retracting of an insertion device.

In some embodiments, the apparatus may be supplied pre-loaded with one or more fixation elements 42. Alternatively, at least one fixation element 42 may be loaded into a fixation device 40 before an intervention, and/or at least one fixation element 42 may be loaded into the fixation device 40 during an intervention.

The fixation device 40 may be capable of fixing a protrusion in a tissue wall in position with regards of the surrounding tissue. The fixation device 40 may be configured to insert a fixation element 42 to secure a first layer of a fold of tissue with regard to a second layer of a fold of tissue, optionally wherein the two layers are fixed so as to be at least partly in contact with one another.

The fixation element 42 may be any suitable means for securing a plication, for example it may be an anchor, additionally or alternatively it may be a suture, optionally a suture secured with a pledget.

Providing a plurality of fixation elements facilitates creating a plurality of plications without removing the apparatus from within a patient and thus may allow for a less time consuming intervention as well as ease of use by an operator.

The fixation element 42 may have a non-deployed state and a deployed state, for example the fixation element 42 may be self-deployable, additionally or alternatively it may be self-expandable.

The non-deployed configuration of a fixation element facilitates the insertion through the plication and the placing of a fixation element. It may further allow for a smaller insertion device.

As illustrated in FIG. 4, in some embodiments the tissue penetrating fixation element 42 may provide a first shoulder 42a for anchoring to, optionally against, a first layer of a tissue plication and may further comprise a second shoulder 42b for anchoring to, optionally against, a second layer of a plication, the first and second shoulders being connected by means of a connecting element 42c extending between the two shoulders 42a and 42b.

In other words the fixation element 42 may be configured to secure the dual tissue walls of a plication and may comprise a first 42a and second 42b shoulder, wherein the first shoulder 42a is configured to secure a first side of tissue and the second shoulder 42b is configured to secure a second side of tissue, further wherein the first and second shoulders 42a and 42b of the fixation element 42 are connected by means of a connecting element 42c, for example, an elastic, optionally a pseudo-elastic.

The first and second shoulders 42a and 42b may be, for example, anchors that connect to the first and second layer of tissue and may further be configured to resist migration from one side to the other. The connective element 42c may extend through a through-aperture from the first layer or side of tissue to the second layer or side of tissue. Additionally or alternatively, the connective element 42c connecting the first and second shoulders, optionally anchors, may allow the securing of the plication by maintaining a predetermined distance between the first shoulder 42a and second shoulder 42b.

The above characteristics help to secure the plication by facilitating the connection between the shoulders 42a and 42b of the fixation element 42 by means of a connective element 42c. The shoulders may help to avoid the migration of the fixation element 42 and the connective element 42c may help avoid the undoing of the plication by facilitating the convergence of a first and second side of a plication.

The connective element 42c may, for example, be an elastic, additionally or alternatively a pseudo-elastic. The connective element 42c may further be biased to a first length; optionally the bias may be maintained so as to bring the dual tissue walls at least partially into a face to face relation for fixing the plication.

In some embodiments the connective element 42c may be longitudinally extendable, optionally wherein the elastic, additionally or alternatively the pseudo-elastic, extension may accommodate a range of tissue thickness at the plication. The longitudinal extension may help avoid injury to tissue, additionally or alternatively may help avoid breakage of the fixation element 42 in the case of excessive forces occurring at the plication.

The longitudinal extension of the connective element 42c may facilitate the adaptation of the plication in function of movements of an organ, for example, it may facilitate the adaptation of a stomach plication during the contractile movements associated with the digestive process.

In some embodiments the fixation element 42 may provide a deployed state and a non-deployed state. In the non-deployed state the first 42a and second 42b shoulders may be rotated, optionally folded, so as to be generally flat. Additionally or alternatively the first and second shoulders, respectively 42a and 42b, may be rotated, optionally folded, to be in line with the connecting element 42c.

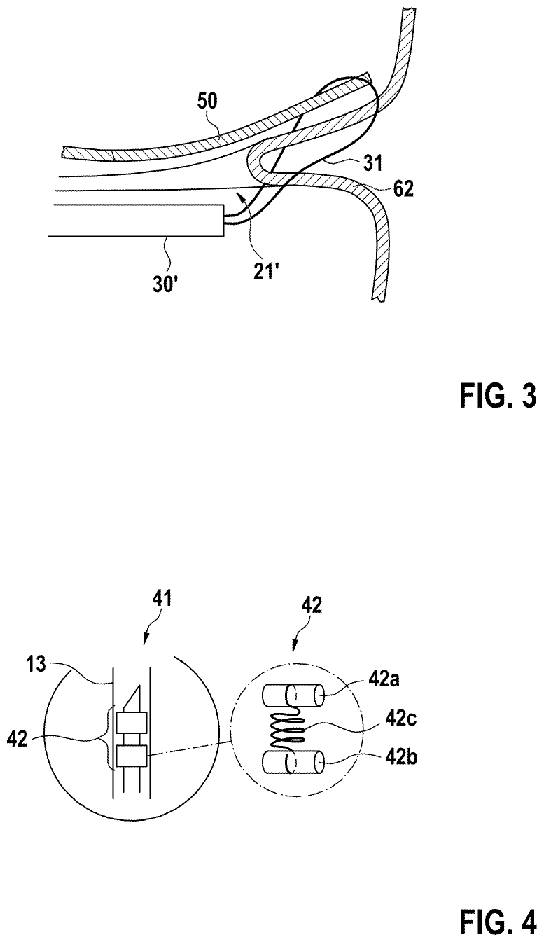

Illustrated in FIG. 3, in some embodiments the apparatus 10 may provide a support shaft 50 for supporting the ligating element 31 of a ligation device 30. The support shaft 50 may extend within, optionally on an instrument 11 and may further extend at least a part of the length of the instrument 11; optionally it may extend the length of the instrument 11. The support 50 may be configured to be adjacent to the ligating device 30. It may be configured to at least temporarily maintain the ligating element 31 of the ligating device 30 in a non-deployed state, optionally wherein the ligating element may circumscribe the traction device 20.

Operating of the operator end 11b may release the ligating element 31 from the support shaft 50, for example, for deployment around a plication.

Further illustrated in FIG. 3, some embodiments may provide a traction device 20 comprising an aspirating or suctioning shaft 21, additionally or alternatively a tapered profile 21a configured for engaging, for example by suctioning, the tissue wall.

The tapered profile 21a of an aspirating device 21 allows suctioning of a predefined amount of tissue so as to form a plication. When aspirated the tissue suctioned may adapt to the tapered profile thus forming a fold in the tissue. When multiple plications are performed, the tapered channel may help facilitate the homogeneity of the plications by suctioning and folding the same amount of tissue for each plication.

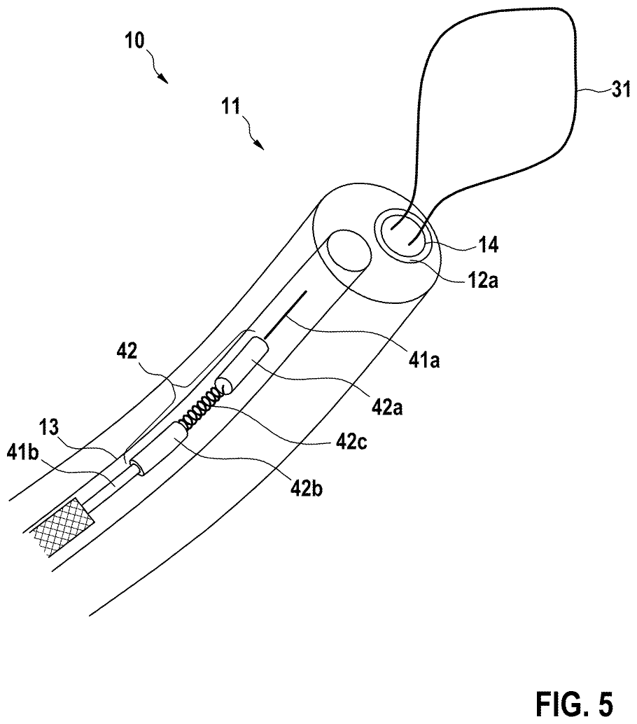

Referring to FIG. 5 some embodiments may provide an apparatus 10 comprising a first channel 12 for accommodating a ligation device 30 and a second channel 13 for accommodating the fixation device 40, wherein the ligation device 30 and the fixation device 40 are at least partly adjacent one another. The insertion device 41 may further provide a tissue penetrating element 41a for traversing a tissue, optionally a tissue plication, additionally or alternatively it may comprise a hollow channel 41b for containing at least one fixation element 42. The tissue penetrating element 41a may be, for example, a needle that may be at least partly flexible. The fixation device 41 may provide multiple fixation elements 42 accommodated within an insertion device 41 for insertion into a tissue to secure a plication.

In some embodiments the apparatus may provide a traction device 20 as disclosed above in combination with a ligating device 30 as disclosed above.

In some embodiments the apparatus may provide a traction device 20 as disclosed above in combination with a fixation device 40 as disclosed above.

In some embodiments the apparatus may provide a traction device 20 as disclosed above in combination with a ligating device 30 as disclosed above and in further combination with a fixation device 40 as disclosed above.

In some embodiments the apparatus may provide a ligation device 30 as disclosed above in combination with a fixation device 40 as disclosed above.

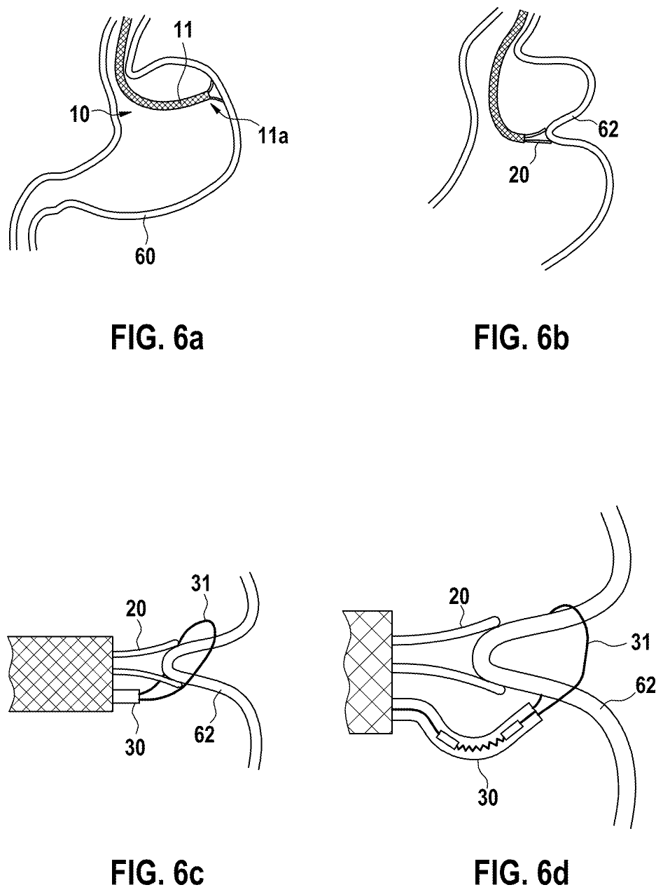

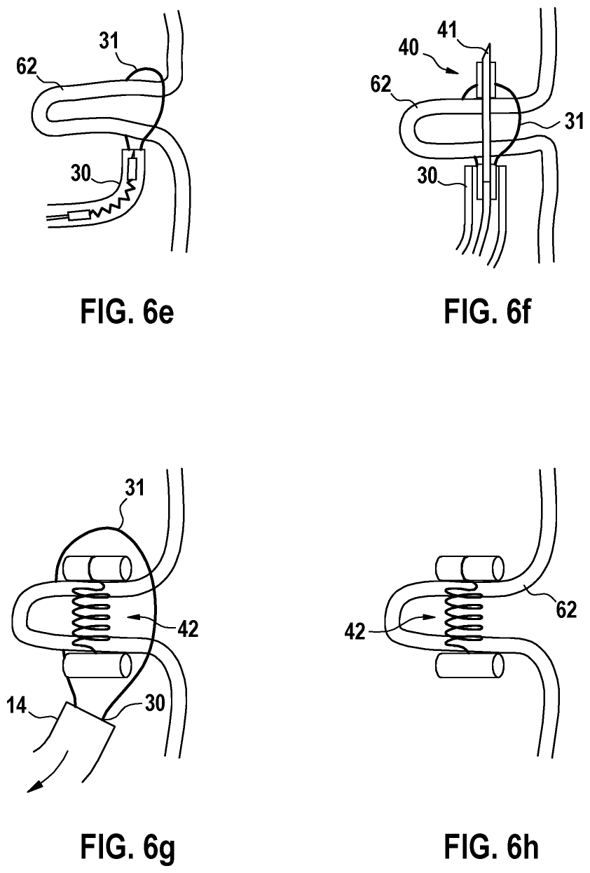

FIGS. 6a-6h illustrate a method of forming a plication in a tissue wall, including a method of operation of the apparatus 10. The drawings illustrate formation of an inward plication in the wall of a patient's stomach 60, but the same principles can be used for other organs or tissue walls.

Referring to FIG. 6a, the working end 11a of the instrument 11 is inserted through the patient's mouth and navigated to the stomach 60 into contact a target site for the plication to be formed in the stomach wall.

Referring to FIG. 6b, the traction device 20 is operated to pull on wall tissue, and to form a protrusion 62 of stomach wall tissue. For example, by applying negative pressure, the tissue wall can be maintained against the traction device, and the traction device then pulled away with respect to the wall.

Referring to FIGS. 6c and 6d, relative movement between (i) the plication device 30 and (ii) the traction device 20 and/or the tissue protrusion 62 causes the plication device to advance over the tissue protrusion 62. For example, the traction device 20 may be retracted, e.g. as part of the pulling to form the protrusion. Additionally or alternatively, the carrier 14 may be advanced to slide the ligating element 31 of the ligating device over the protrusion 62.

Referring to FIG. 6e, the ligating device 30 is operated to tighten the ligating element 31 around the protrusion 62. For example, the ligation element 31 serves to immobilize the protrusion 62, optionally allowing the traction device 20 to be disengaged from the tissue. Also for example, the ligating element 31 serves to laterally com-press the protrusion 62 to form or define the plication in the tissue wall.

Referring to FIG. 6f, the fixation device 40 is operated to begin fixing the plication in the wall tissue. For example, the insertion device 41 can be extended from the carrier 14 to penetrate through the overlaid tissue wall portions adjacent to and/or held by the ligating device 30. The insertion device 41 can be progressively retracted to controllably deploy the tissue-penetrating fixation element 42 (FIG. 6g) to complete fixing the plication at that point. Thereafter, the ligating device 30 can be disengaged (FIGS. 6g and 6h), for example, by relaxing the ligating element 31, and retracting the carrier 14 away from the protrusion.

The process can be repeated, as many times as appropriate, at one or more neighbouring target sites, to extend or elongate the plication in a certain direction. The fixation device 40 can be loaded with multiple fixation elements 42 to enable the process to be repeated without having to remove the instrument from the patient's stomach.

Generally, the invention may thus extend to a method of operation of an apparatus, optionally as described above, for forming at least one plication in a wall of an internal organ of a patient and wherein the apparatus may comprise an elongated instrument 11 for insertion into a patient.

The method may comprise a first step (a) for operating a traction device 20 to engage with tissue of an organ wall and to pull on said tissue of the organ wall to create a protrusion of organ wall tissue.

Additionally or alternatively, the method may comprise a second step (b) for operating a ligating device 30 to circumscribe the, optionally a, protrusion of a tissue wall to define, additionally or alternatively to form, a plication in the tissue wall.

Further additionally or alternatively the method may provide a third step (c) for operating a fixation device 40 to fix the plication in position.

Specific aspects of the invention may provide a combination of steps (a) and (b); additionally or alternatively a combination of (b) and (c); additionally or alternatively a combination of (a) and (c); additionally or alternatively a combination of (a), (b) and (c).

Also generally, the invention may extend to a method for creating a plication in a wall of an internal organ of a patient, optionally using an apparatus as described above.

The method may comprise or further comprises a first step (i) for inserting an instrument 11 into the patient through a natural or non-natural opening, to the organ; and at least one or more of the following steps:

A second step (ii) of engaging tissue of the organ wall, and pulling on the tissue to create a protrusion of organ wall tissue.

Additionally or alternatively, the method may provide a third step (iii) of ligating the, optionally a, protrusion of organ wall tissue, to circumscribe the protrusion and define additionally or alternatively to form at least one plication in an organ wall.

Further additionally or alternatively the method may comprise a fourth step (iv) of fixing the plication using a tissue-penetrating fixation element.

In a fourth aspect the invention may provide a method for creating a plication in a wall of an internal organ of a patient the method may optionally use the apparatus as described in any of the above aspects and may provide an elongated instrument for insertion into a patient. The method may comprise one or more of the following steps (1) to (8).

The method may provide a first step (1) for inserting an elongated instrument 11 into the patient by means of an access site and guiding the instrument 11 to the desired location for the plication.

Additionally or alternatively, the second step (2) for engaging a traction device 20 with the organ wall and capturing a portion of the organ wall, for example by activating a suctioning element 21.

Additionally or alternatively, the method may comprise a further step (3) for pulling the captured tissue so as to create a fold, for example, by aspirating a portion of tissue.

Further additionally or alternatively, the method may provide a step (4) for deploying a ligating device 30 around the fold and tightening the ligating element 31 so as to temporarily secure the fold.

The method may comprise an additional or alternative step (5) for releasing the traction device 20 engaging the organ wall.

An additional or alternative step (6) may be provided by the method, for delivering a fixing element 42 to permanently or at least semi-permanently secure the plication, the delivering of a fixing element may be facilitated by penetrating an internal tissue by an insertion device 41.

The method may further provide an additional or alternative step (7) for pivoting the apparatus 10 and repeating steps (2) to (6) if multiple plications are desired, for example, to create an elongated plication.

An additional or alternative final step (8) may be provided for retracting the apparatus 10 from within the patient.

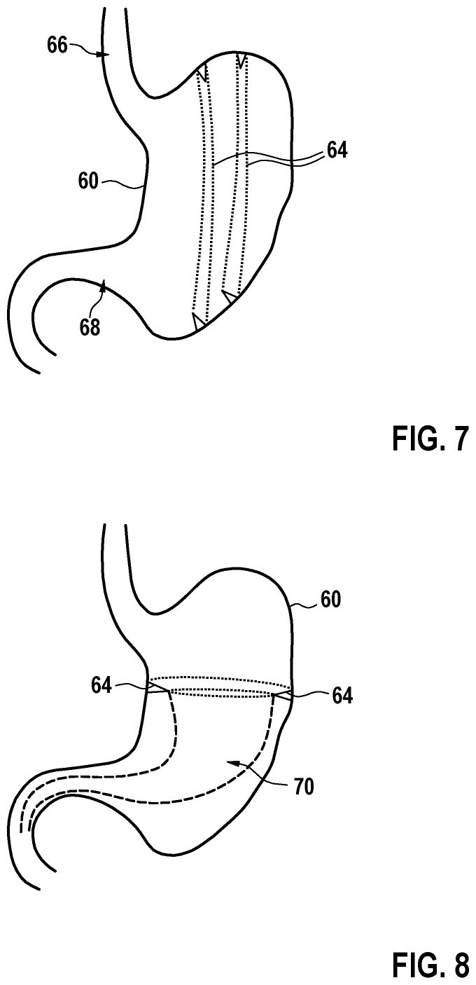

FIGS. 7 and 8 illustrate examples of elongated plications in wall tissue, optionally created using the apparatus and/or method(s) described herein. The drawings illustrate plications of the stomach 60 for the treatment of diabetes or obesity of a patient, for example by modifying one or more of stomach volume, appetite saiety, and digestion by the body.

FIG. 7 illustrates plications 64 extending lengthwise within the stomach 60, to narrow the stomach cavity, and/or reduce the stomach volume, and/or change the stomach form into a more streamlined sleeve shape in a direction from the stomach entrance 66 to the stomach exit 68.

FIG. 8 illustrates an annular plication 64 that can define a mounting site, such as a seat, and/or dock, and/or a reinforced tissue fold, for a functional device 70 insertable at least partly in the stomach 60. The functional device 70 may, for example, be a bypass sleeve. The sleeve may, for example, function to reduce the active volume of the stomach 60, and to guide digested or partly digested food into and/or at least partly through the small intestine. The sleeve 70 may extend at least partly into and/or through the duodenum, and optionally at least partly into the jejunum. Additionally or alternatively, other functional devices 70 may be mounted using such a plication 64 in the stomach wall, for example, a functional device to occupy stomach volume, or a functional device to reduce stomach volume, or affect downstream digestion.

It will be appreciate that the foregoing description is merely illustrative of example forms of the invention, and that many modifications and equivalents can be used within the principles of the invention.

1.-18. (canceled)

19. An apparatus for forming a plication in a wall of internal tissue of a patient, the apparatus comprising an elongated instrument for insertion into a patient close to the tissue wall, wherein the apparatus comprises one or more of:
(a) a traction device configured to engage tissue of the wall and to pull on tissue of the wall to create a protrusion of wall tissue;
(b) a ligating device configured to at least partially circumscribe the or a protrusion in tissue of the wall to form the plication in the tissue wall; and
(c) a fixation device for fixing the plication in the tissue wall.

20. The apparatus according to claim 19, wherein the ligating device comprises a ligating element which circumscribes the traction device in a circumferential direction, at least in one operative condition of one or more of the ligating device or of the traction device.

21. The apparatus according to claim 19, wherein the fixation device comprises one or more of a tissue-penetrating fixation element or an insertion device for inserting the fixation element, the fixation element being configured to extend through the wall tissue at the plication.

22. The apparatus according to claim 19, wherein the traction device extends through a loop of the ligating device, at least in one operative condition of one or more of the ligating device or the traction device.

23. The apparatus according to claim 19, wherein the traction device comprises a suction or aspiration device for pulling on a wall tissue by application of negative pressure.

24. The apparatus according to claim 21, wherein the insertion device and the ligating device are carried on a same carrier.

25. The apparatus according to claim 21, wherein the ligating device and the insertion device are arranged on a carrier adjacent one another for interacting with the same region, or neighbouring regions, of tissue of the wall.

26. The apparatus according to claim 19, wherein the instrument further comprises an elongated main body having a working end for contacting the tissue wall, and an operator end from which one or more of: the traction device, the ligating device or the insertion device may be operated.

27. The apparatus according to claim 26, wherein the main body comprises a first channel for one or more of: accommodating the traction device or through which the traction device may be operated, and a second channel for accommodating one or more of a carrier tube for the ligating device or the insertion device.

28. The apparatus according to claim 21, wherein the tissue-penetrating fixation element has a first shoulder for anchoring against a first side of tissue, a second shoulder for anchoring against a second side of tissue, and a connecting element extending between the first shoulder and the second shoulder.

29. The apparatus according to claim 28, wherein the connecting element is one or more of elastic or pseudoelastic.

30. The apparatus according to claim 28, wherein the fixation element is insertable into the second channel in a non-deployed configuration in which the first shoulder and the second shoulder are configured in a position with a smaller outer diameter,

31. A method of operation of an apparatus for forming a plication in a wall of internal tissue of a patient, the apparatus comprising an elongated instrument for insertion into a patient, the method comprising one or more of:
(a) operating a traction device to engage tissue of the wall and to pull on tissue of the wall to create a protrusion of wall tissue;
(b) operating a ligating device to circumscribe a protrusion in wall tissue to form the plication in the wall; and
(c) operating a fixation device to fix the plication in position.

32. The method for forming a plication in a wall of internal tissue of a patient using an apparatus according to claim 19, the method comprising:
(i) inserting an instrument into the patient to the tissue wall through a natural or non-natural opening, and one or more of the following:
(ii) engaging tissue of the wall, and pulling on the tissue to create a protrusion of wall tissue;
(iii) ligating a protrusion of wall tissue, to circumscribe the protrusion and form the plication in the tissue wall; and
(iv) fixing the plication using a tissue-penetrating fixation element.

33. The method according to claim 31, further including releasing a pulling device from the tissue wall.

34. The method according to claim 32, further including releasing a pulling device from the tissue wall.

35. A method of placing a functional device in the stomach of a patient, the method comprising:
(i) forming a plication in wall tissue of the stomach, the plication serving as a mounting site for the functional device; and
(ii) placing the functional device such that the functional device is mounted with respect to the stomach using, at least partly, the plication.

36. An apparatus for placing a functional device within a stomach of a patient, the apparatus comprising:
(i) a first apparatus for forming a plication in wall tissue of the stomach, the plication serving as a mounting site for the functional device; and
(ii) a second apparatus for placing the functional device such that the functional device is mounted with respect to the stomach using, at least partly, the plication.

37. A functional device for placement at least partly within a stomach of a patient, wherein the functional device comprises a mounting portion configured to be mounted to a plication formed in wall tissue of the stomach, whereby the functional device is mountable with respect to the stomach using, at least partly, the plication.

38. The functional device according to claim 37, wherein the functional device is or comprises a bypass sleeve.
